(19) United States
(12) Patent Application Publication
(10) Pub. No.: US 2015/0025143 A1
(43) Pub. Date: Jan. 22, 2015

(54) BETA-HYDROXY-BETA-METHYLBUTYRIC ACID FOR IMPROVING GLUCOSE TOLERANCE

(71) Applicant: ABBOTT LABORATORIES, ABBOTT PARK, IL (US)

(72) Inventors: Shreeram Sathyavageeswaran, Singapore (SG); Tapas Das, Worthington, OH (US); Srabani Das, Singapore (SG)

(73) Assignee: ABBOTT LABORATORIES, ABBOTT PARK, IL (US)

(21) Appl. No.: 14/386,508

(22) PCT Filed: Mar. 18, 2013
(86) PCT No.: PCT/US2013/032831

§ 371 Date: Sep. 19, 2014

Related U.S. Application Data
(60) Provisional application No. 61/612,634, filed on Mar. 19, 2012.

(51) Int. Cl. A61K 31/19; A23L 1/29

(52) U.S. Cl. CPC A61K 31/19; A23L 1/296; A23V 2002/00
USPC 514/557

(57) ABSTRACT

The use beta-hydroxy-beta-methylbutyric acid for improving glucose tolerance in a pediatric individual or in an adult individual is disclosed. In certain embodiments, the beta-hydroxy-beta-methylbutyric acid is administered via a nutritional composition.

### CROSS-REFERENCE TO RELATED APPLICATION

This application claims priority to and any other benefit of U.S. Provisional Patent Application Ser. No. 61/612,634, filed Mar. 19, 2012 and entitled “BETA-HYDROXY-BETA-METHYLBUTYRIC ACID FOR IMPROVING GLUCOSE TOLERANCE,” the entire disclosure of which is incorporated by reference herein.

### TECHNICAL FIELD

The present disclosure relates to nutritional compositions including beta-hydroxy-beta-methylbutyric acid and methods for improving glucose tolerance in an individual using the nutritional compositions. More specifically, the present disclosure relates to pediatric nutritional compositions and methods that include or utilize beta-hydroxy-beta-methylbutyric acid (HMB) for improving glucose tolerance in pediatric individuals and adult individuals.

### BACKGROUND OF THE DISCLOSURE

In a person with normal metabolism, insulin is released from the beta cells of Islets of Langerhans located in the pancreas in response to an elevated blood glucose level, allowing glucose to enter insulin-sensitive tissues and maintain normal blood glucose levels. Diabetes has become the fourth leading cause of death in most developed countries and will be one of the most challenging health problems worldwide in the 21st century.

There are two major forms of diabetes: Type-1 diabetes is characterized by the inability to synthesize insulin and Type-2 diabetes is characterized by the body becoming resistant to the effects of insulin. The beta cell dysfunction increases basal insulin secretion, but impairs glucose stimulated insulin secretion. In an “insulin resistant” (also referred to herein as a “prediabetic”) individual, the body is less sensitive to insulin levels in the blood, and hence the metabolic activities triggered by insulin as seen in normal individuals do not proceed or proceed at lower levels. This leads to a condition in which normal amounts of insulin are inadequate to produce a normal insulin response from fat, muscle and liver cells, i.e. the cells are not able to absorb glucose (i.e., glucose intolerance) and other nutrients.

As a result of the lowered metabolic response, the normal physiological feedback mechanisms cause the beta cells to increase insulin production to compensate for the insensitivity of the response to insulin. As the insulin response continues to decrease, insulin production continues to increase. However, sustained insulin resistance weakens the beta cells and gradually degrades the insulin secretion capacity, thus proceeding to a more pronounced diabetic stage.

Prediabetes may be detectable in an individual as early as 20 years before diabetic symptoms become evident in that individual. Studies have shown that although patients may show very few symptoms, long-term physiological damage is already occurring at this stage. Up to 60% of these individuals will progress to type 2 diabetes within 10 years.

As such, there is a need for nutritional compositions and methods for improving glucose tolerance in individuals generally, and particularly in pediatric individuals and other potential prediabetics, to prevent or delay the development of diabetes. It would additionally be beneficial if the nutritional compositions could be used for treating and/or managing and/or controlling and/or reducing insulin resistance and glucose intolerance, as well as their related metabolic defects including hyperglycemia.

### SUMMARY OF THE DISCLOSURE

The present disclosure is directed to nutritional compositions including beta-hydroxy-beta-methylbutyric acid and to methods of using the compositions to improve glucose intolerance and glucose metabolism in an individual, including pediatric individuals, adult individuals, and older adult individuals. The compositions and methods of the present disclosure may be particularly beneficial for pediatric, adult, and older adult individuals who have a family history of diabetes as the nutritional compositions and methods may delay or prevent altogether the onset of diabetes.

One embodiment of the present disclosure is directed to a pediatric nutritional composition comprising at least one of fat, protein, carbohydrate and from about 0.1% to about 2.0% beta-hydroxy-beta-methylbutyric acid by weight.

Another embodiment of the present disclosure is directed to a method for improving glucose tolerance in a pediatric individual in need thereof. The method comprises administering to the pediatric individual a composition comprising an effective amount of beta-hydroxy-beta-methylbutyric acid.

Another embodiment of the present disclosure is directed to a pediatric nutritional composition comprising calcium beta-hydroxy-beta-methylbutyric acid, whey protein, casein protein, soy protein, medium chain triglyceride oil, and fructooligosaccharides.

Another embodiment of the present disclosure is directed to a method for improving glucose tolerance in an adult individual in need thereof. The method comprises administering to the individual a composition comprising an effective amount of beta-hydroxy-beta-methylbutyric acid.

It has now been discovered that beta-hydroxy-beta-methylbutyric acid can be utilized to improve glucose tolerance and improve glucose metabolism in individuals including adults, older adults, and pediatric individuals. Significantly, it has been found that beta-hydroxy-beta-methylbutyric acid plays a role in both improving glucose metabolism and improving the glycemic response; both of which can be instrumental in delaying the onset of conditions and diseases related to glucose intolerance. Additionally, beta-hydroxy-beta-methyl butyric acid may further be used to prevent, treat, reduce, control and/or manage glucose intolerance, hyperglycemia and diabetes in individuals including adults, older adults, and pediatric individuals.

Accordingly, the beta-hydroxy-beta-methylbutyric acid-containing nutritional compositions and methods of the present disclosure offer an alternative therapeutic option that may contribute to improved glucose tolerance in individuals, and particularly pediatric individuals. These benefits are advantageously achieved without the complications seen with the previously used oral synthetic pharmacological approaches.

### BRIEF DESCRIPTION OF THE DRAWINGS

FIG. 1 is a graph depicting the effects of HMB on oral glucose tolerance as evaluated in Example 1.

FIG. 2 is a graph depicting the effect of calcium HMB on lowering glycemic index as evaluated in Example 2.

### DETAILED DESCRIPTION OF THE DISCLOSURE

The nutritional compositions described herein, along with their methods of use, include beta-hydroxy-beta-methylbutyric acid for improving glucose tolerance and related conditions and diseases, particularly in the pediatric and adult populations. Glucose intolerance and diminished glucose metabolism in many individuals ultimately leads to diabetes, and in some cases, severe diabetes that can significantly impact the quality of life for the individual. The nutritional compositions and related methods as described herein provide individuals, and particularly pediatric individuals, with a method of improving glucose tolerance and improving glucose metabolism early in life so that a healthy balance can be maintained and diabetes and diabetic conditions may be altogether avoided, or at least reduced. The nutritional compositions and methods as described herein may provide a pediatric individual who is at risk for glucose tolerance issues, including diabetes, with methods for preventing, or at a minimum, delaying the onset of diabetes through nutritional intervention.

The elements or features of the various embodiments are described in detail hereinafter.

The terms “retort” and “retort sterilized” are used interchangeably herein, and unless otherwise specified, refer to the common practice of filling a container, most typically a metal can or other similar package, with a nutritional liquid, such as a liquid pediatric formula, and then subjecting the liquid-filled package to the necessary heat sterilization step, to form a retort sterilized nutritional liquid product.

The terms “improve glucose tolerance” as used herein, unless otherwise specified, means an improvement in an individual's ability to properly metabolize glucose in the body such that the glucose is more efficiently used in the body.

The term “older adult” as used herein, unless otherwise specified, refers to an individual 50 years old or older.

The terms “aseptic” and “aseptic sterilized” are used interchangeably herein, and unless otherwise specified, refer to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The terms “nutritional formula” or “nutritional product” or “nutritional composition,” as used herein, are used interchangeably and, unless otherwise specified, refer to nutritional liquids, nutritional semi-liquids, nutritional semi-solids, nutritional powders, nutritional supplements, and any other nutritional food product as known in the art. The nutritional powders may be reconstituted to form a nutritional liquid, all of which comprise one or more of fat, protein and carbohydrate, and are suitable for oral consumption by a human.

The term “nutritional liquid,” as used herein, unless otherwise specified, refers to nutritional products in ready-to-drink liquid form, concentrated form, and nutritional liquids made by reconstituting the nutritional powders described herein prior to use.

The term “nutritional powder,” as used herein, unless otherwise specified, refers to nutritional products in flowable or scoopable form that can be reconstituted with water or another aqueous liquid prior to consumption and includes both spray dried and drymixed/dryblended powders.

The terms “pediatric” or “pediatric individual” are used herein interchangeably to refer to individuals from the age of greater than 1 year to 12 years, including the age of greater than 1 year to 10 years.

The term “pediatric nutritional composition,” as used herein, refers to nutritional products that are designed specifically for consumption by a pediatric individual.

The terms “fat,” “oil” and “lipid” as used herein, unless otherwise specified, are used interchangeably to refer to lipid materials derived or processed from plants or animals. These terms also include synthetic lipid materials so long as such synthetic materials are suitable for oral administration to humans.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

All numerical ranges as used herein, whether or not expressly preceded by the term “about”, are intended and understood to be preceded by that term, unless otherwise specified.

The nutritional compositions and methods herein may also be free of any optional or other ingredient or feature described herein provided that the remaining composition still contains the requisite ingredients or features as described herein. In this context, the term “free” means the selected composition or method contains or is directed to less than a functional amount of the ingredient or feature, typically less than 0.1% by weight, and also including zero percent by weight, of such ingredient or feature.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any reference to a singular characteristic or limitation of the present disclosure shall include the corresponding plural characteristic or limitation, and vice versa, unless otherwise specified or clearly implied to the contrary by the context in which the reference is made.

Any combination of method or process steps as used herein may be performed in any order, unless otherwise specifically or clearly implied to the contrary by the context in which the referenced combination is made.

The nutritional compositions and methods may comprise, consist of, or consist essentially of the elements and features of the disclosure described herein, as well as any additional or optional ingredients, components, or features described herein or otherwise useful in a nutritional application.

### Product Form

The nutritional compositions of the present disclosure include beta-hydroxy-beta-methylbutyric acid and include both adult and pediatric nutritional compositions. The nutritional compositions may be formulated and administered in any known or otherwise suitable oral product form. Any solid, semi-solid, liquid, semi-liquid, or powder form, including combinations or variations thereof, are suitable for use herein, provided that such forms allow for safe and effective oral delivery to the individual of the ingredients as also defined herein.

The nutritional compositions of the present disclosure include any product form comprising the ingredients described herein, and which is safe and effective for oral administration. The nutritional compositions may be formulated to include only the ingredients described herein, or may be modified with optional ingredients to form a number of different product forms. The nutritional compositions of the present disclosure are preferably formulated as dietary product forms, which are defined herein as those embodiments comprising the ingredients of the present disclosure in a product form that then contains at least one of fat, protein, and carbohydrate, and preferably also contains vitamins, minerals, or combinations thereof.

The nutritional compositions of the present disclosure may therefore include a variety of different product forms, including most any conventional or otherwise known food product form, some non-limiting examples of which include confectionary products, cereals, food condiments (e.g., spreads, powders, sauces, jams, jelly, coffee creamer or sweetener), pasta, baking or cooking materials (e.g., flour, fats or oils, butter or margarine, breading or baking mixes), salted or seasoned snacks (e.g., extruded, baked, fried), beverages (e.g., coffee, juice, carbonated beverage, non-carbonated beverage, tea, ice-cream based drinks), snack or meal replacement bars (e.g., Slimfast™ bars, Ensure™ bars, Zone perfect™ bars, Glucerna™ bars), smoothies, breakfast cereals, cheeses, gummy products, salted or unsalted crisp snacks (e.g., chips, crackers, pretzels), dips, baked goods (e.g., cookies, cakes, pies, pastries, bread, bagels, croutons, dressings, dry mixes (e.g., mixes for muffins, cookies, waffles, pancakes, beverages)), frozen desserts (e.g., ice cream, popsicles, fudge bars, crushed ice, frozen yogurt), pasta, processed meats (e.g., corn dogs, hamburgers, hotdogs, sausage, pepperoni), pizza, pudding, flavored or unflavored gelatin, refrigerated dough (e.g., cookies, bread, brownies), milk or soy-based smoothies, yogurt or yogurt-based drinks, frozen yogurt, soy milk, soups, vegetable-based burgers, and popcorn-based snacks.

The nutritional compositions of the present disclosure may also be formulated in product forms such as capsules, tablets, pills, caplets, gels, liquids (e.g., suspensions, solutions, emulsions, clear solutions), powders or other particulates, and so forth. These product forms generally contain only the ingredients as described herein, optionally in combination with other actives, processing aids or other dosage form excipients.

The nutritional compositions of the present disclosure, when formulated as a dietary product form, may potentially provide either a sole source or a supplemental source of nutrition to an individual. In this context, a sole source of nutrition is one that can be administered once or multiple times each day to potentially provide an individual with all or substantially all their fat, protein, carbohydrate, mineral, and vitamin needs per day or during the intended period of administration. A supplemental source of nutrition is defined herein as a dietary source that does not provide an individual with a potentially sole source of nutrition.

The nutritional compositions of the present disclosure are desirably formulated as milk-based liquids, soy-based liquids, low-pH liquids, clear liquids, reconstitutable powders, nutritional bites (e.g., plurality of smaller dietary product dosage forms in a single package), or nutritional bars (snack or meal replacement).

### Beta-Hydroxy-Beta Methylbutyric Acid (HMB)

The nutritional compositions of the present disclosure comprise HMB, which means that the nutritional compositions are either formulated with the addition of HMB, most typically as a calcium monohydrate, or are otherwise prepared so as to contain HMB in the finished product. Any source of HMB is suitable for use herein provided that the finished product contains HMB, although such a source is preferably calcium HMB and is most typically added as such to the nutritional products during formulation.

Although calcium HMB monohydrate is the preferred source of HMB for use herein, other suitable sources may include HMB as the free acid, a salt, an anhydrous salt, an ester, a lactone, or other product forms that otherwise provide a bioavailable form of HMB from the nutritional product. Non-limiting examples of suitable salts of HMB for use herein include HMB salts, hydrated or anhydrous, of sodium, potassium, magnesium, chromium, calcium, or other non-toxic salt form. Calcium HMB monohydrate is preferred and is commercially available from Technical Sourcing International (TSI) of Salt Lake City, Utah and from Lonza Group Ltd. (Basel, Switzerland).

The nutritional compositions as described herein include an amount of HMB that is sufficient and effective to improve an individual's, and specifically a pediatric individual's, glucose tolerance; that is, the nutritional compositions described herein include a sufficient amount of HMB to allow an individual, and desirably a pediatric individual, to improve glucose metabolism.

When the nutritional product is a liquid, the concentration of HMB in the liquid may range up to 10%, including from about 0.1% to about 8%, and also including from about 0.1% to about 2%, and also including from about 0.1% to about 5%, and also including from about 0.3% to about 3%, and also including from about 0.34% to about 1.5%, by weight of the nutritional liquid. In one specific embodiment the HMB is present in the liquid formulation in an amount of from about 0.1% to about 0.5% by weight of the nutritional liquid.

When the nutritional product is a solid, the concentration of HMB in the solid may range up to 15%, including from about 0.1% to about 10%, and also including from about 0.1% to about 2% and also including from about 0.2% to about 5%, and also including from about 0.3% to about 3%, and also including from about 0.34% to about 1.5%, by weight of the nutritional powder. In a specific embodiment, the HMB is present in the powder formulation in an amount of from about 0.1% to about 0.5% by weight of the nutritional powder.

### Macronutrients

The nutritional compositions of the present disclosure may further comprise one or more optional macronutrients in addition to the HMB described herein. The optional macronutrients include proteins, fats, carbohydrates, and combinations thereof The nutritional compositions are desirably formulated as dietary products containing all three macronutrients.

Macronutrients suitable for use herein include any protein, fat, or carbohydrate or source thereof that is known for or otherwise suitable for use in an oral nutritional composition, provided that the optional macronutrient is safe and effective for oral administration and is otherwise compatible with the other ingredients in the nutritional composition.

The concentration or amount of optional fat, carbohydrate, and protein in the nutritional composition can vary considerably depending upon the particular product form (e.g., bars or other solid dosage forms, milk or soy based liquids or other clear beverages, reconstitutable powders, etc.) and the various other formulations and targeted dietary needs. These optional macronutrients are most typically formulated within any of the embodied ranges described in the following tables.

TABLE 1

| Nutrient (% total calories) | Example A | Example B | Example C |
|---|---|---|---|
| Carbohydrate | 0-100 | 10-70 | 40-50 |
| Fat | 0-100 | 20-65 | 35-55 |
| Protein | 0-100 | 5-40 | 15-25 |

Each numerical value preceded by the term “about”

TABLE 2

| Nutrient (wt % composition) | Example D | Example E | Example F |
|---|---|---|---|
| Carbohydrate | 0-98 | 1-50 | 10-30 |
| Fat | 0-98 | 1-30 | 1-15 |
| Protein | 0-98 | 1-30 | 1-10 |

Each numerical value preceded by the term “about”

### Carbohydrate

Carbohydrates suitable for use in the nutritional compositions may be simple, complex, or variations or combinations thereof, all of which are optionally in addition to the HMB as described herein. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Carbohydrates suitable for use herein also include soluble dietary fiber, non-limiting examples of which include gum Arabic, fructooligosaccharide (FOS), sodium carboxymethyl cellulose, guar gum, citrus pectin, low and high methoxy pectin, oat and barley glucans, carrageenan, psyllium and combinations thereof. Insoluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof

The nutritional compositions may therefore, and desirably, further comprise a carbohydrate in addition to the HMB, wherein for solid embodiments of the nutritional compositions of the present disclosure, the solid embodiments generally comprise carbohydrates in addition to the HMB in quantities ranging up to 75%, including from about 20% to about 70%, and also including from about 50% to about 70%, and also including from about 55% to about 65%, and also including from about 58% to about 62%, by weight of the solid nutritional composition.

For liquid embodiments of the nutritional compositions of the present disclosure, the liquid embodiments generally comprise carbohydrate in addition to the HMB in quantities ranging up to 30%, including from about 5% to about 25%, and also including from about 10% to about 20%, and also including from about 15% to about 18%, by weight of the liquid nutritional composition.

### Protein

Proteins suitable for use in the nutritional compositions include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The nutritional compositions of the present disclosure may optionally comprise a soy protein component, sources of which include, but are not limited to, soy flakes, soy protein isolates, soy protein concentrate, hydrolyzed soy protein, soy flour, soy protein fiber, or any other protein or protein source derived from soy. Commercial sources of soy protein are well known in the nutrition art, some non-limiting examples of which include soy protein isolates distributed by The Solae Company (St. Louis, Mo.) under the trade designation “Soy Protein Isolate EXP-H0118,” “EXP-E-0101, and “Supro Plus 675.”

The optional soy protein component may represent from zero to 100%, desirably from about 10% to 100%, and including from about 15% to 100%, and also including from about 75% to about 95%, and also including from about 80% to about 90% of the total protein calories in the composition.

The nutritional compositions may therefore, and desirably, further comprise a protein in addition to the HMB, wherein for solid embodiments of the nutritional compositions of the present disclosure, the solid embodiments generally comprise protein in addition to the HMB in quantities ranging up to 30%, including from about 5% to about 25%, and also including from about 10% to about 20%, and also including from about 12% to about 16%, by weight of the solid nutritional composition.

For liquid embodiments of the nutritional compositions of the present disclosure, the liquid embodiments generally comprise protein in quantities ranging up to 30%, including from about 1% to about 20%, and also including from about 1% to about 10%, and also including from about 5% to about 8%, by weight of the liquid nutritional composition.

### Fat

Fats suitable for use in the nutritional compositions include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, marine oils, flaxseed oil, borage oil, cottonseed oils, evening primrose oil, blackcurrant seed oil, transgenic oil sources, fungal oils, marine oils (e.g., tuna, sardine), and so forth.

The nutritional compositions of the present disclosure optionally comprise a flaxseed component, non-limiting examples of which include ground flaxseed and flaxseed oil. Ground flaxseed is generally preferred. Non-limiting examples of flaxseed include red flaxseed, golden flaxseed, and combinations thereof. Golden flaxseed is generally preferred. Commercial sources of flaxseed are well known in the nutrition and formulation arts, some non-limiting examples of which include flaxseed and flax products available from the Flax Council of Canada, the Flax Consortium of Canada, and Heintzman Farms (North Dakota) (Dakota Flax Gold brand).

The nutritional compositions may therefore, and desirably, further comprise a fat in addition to the HMB, wherein for solid embodiments of the nutritional compositions of the present disclosure, the solid embodiments generally comprise fat in addition to the HMB in quantities ranging up to 35%, including from about 5% to about 30%, and also including from about 10% to about 25%, and also including from about 15% to about 20%, by weight of the solid nutritional composition.

For liquid embodiments of the nutritional compositions of the present disclosure, the liquid embodiments generally comprise fat in addition to the HMB in quantities ranging up to 30%, including from about 1% to about 20%, and also including from about 1% to about 10%, and also including from about 5% to about 9%, by weight of the liquid nutritional composition.

Other Optional Ingredients

The nutritional compositions of the present disclosure may further comprise other optional components that may modify the physical, chemical, aesthetic or processing characteristics of the compositions or serve as pharmaceutical or additional nutritional components when used in the targeted population. Many such optional ingredients are known or otherwise suitable for use in nutritional compositions or pharmaceutical dosage forms and may also be used in the compositions herein, provided that such optional ingredients are safe and effective for oral administration and are compatible with the other selected ingredients in the composition.

Non-limiting examples of such other optional ingredients include preservatives, anti-oxidants, buffers, additional pharmaceutical actives, sweeteners including artificial sweeteners (e.g., saccharine, aspartame, acesulfame K, sucralose), colorants, flavors, branch chain amino acids, essential amino acids, free amino acids, flavor enhancers, thickening agents and stabilizers, emulsifying agents, lubricants, and so forth.

The nutritional compositions of the present disclosure preferably comprise one or more minerals, non-limiting examples of which include phosphorus, sodium, chloride, magnesium, manganese, iron, copper, zinc, iodine, calcium, potassium, chromium (e.g., chromium picolinate), molybdenum, selenium, and combinations thereof.

The nutritional compositions also desirably comprise one or more vitamins, non-limiting examples of which include carotenoids (e.g., beta-carotene, zeaxanthin, lutein, lycopene), biotin, choline, inositol, folic acid, pantothenic acid, choline, vitamin A, thiamine (vitamin B1), riboflavin (vitamin B2), niacin (vitamin B3), pyridoxine (vitamin B6), cyanocobalamine (vitamin B12), ascorbic acid (vitamin C), vitamin D, vitamin E, vitamin K, and various salts, esters or other derivatives thereof, and combinations thereof In some preferred embodiments, the nutritional compositions of the present disclosure comprise both vitamins and minerals.

### Methods of Using the HMB-Containing Nutritional Compositions

The nutritional compositions including HMB as described herein can be used in various methods as set forth herein for individuals, including adults, older adults, and pediatric individuals. These methods include the oral administration of the beta-hydroxy-beta-methylbutyric acid-containing nutritional compositions to the individual to improve glucose tolerance and related conditions. Additionally, the nutritional compositions may be administered to improve glucose metabolism generally in the body, including glucose metabolism in muscles, and to treat and/or prevent and/or control and/or manage and/or reduce glucose intolerance, hyperglycemia and/or diabetes, by which is meant that the methods may be used in individuals afflicted by (generally prediabetics or diabetics) or otherwise at risk of, or susceptible to, (generally obese individuals or individuals with a family history) developing glucose intolerance, hyperglycemia and/or diabetes (an individual in need of administration of the HMB-containing nutritional composition).

In many embodiments, the methods can be used to slow the onset or progression of glucose intolerance, hyperglycemia and/or diabetes and can be used to reverse the effects of glucose intolerance, hyperglycemia, and/or diabetes in individuals, including pediatric individuals. The methods include administration of the nutritional compositions to individuals, including pediatric individuals specifically, in need thereof, including individuals, including pediatric individuals specifically, afflicted with glucose intolerance, hyperglycemia or diabetes, and/or individuals, including pediatric individuals specifically, at risk of developing glucose intolerance, hyperglycemia or diabetes due to heredity or other factors. As such, in some embodiments of the present disclosure, the methods disclosed herein are directed to a subset of the general population, including the older adults and the general pediatric population, such that in these embodiments not all of the general population can benefit from these methods.

The individual desirably consumes at least one serving of the nutritional composition daily, and in some embodiments, may consume two, three, or even more servings per day. Each serving is desirably administered as a single, undivided dose, although the serving may also be divided into two or more partial or divided servings to be taken at two or more times during the day. The methods of the present disclosure include continuous day after day administration, as well as periodic or limited administration, although continuous day after day administration is generally desirable. The methods of the present disclosure are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least 18-24 months, desirably as a long term, continuous, daily, dietary supplement.

The methods of the present disclosure as described herein are also intended to include the use of such methods in individuals unaffected by or not otherwise afflicted with hyperglycemia, glucose intolerance, etc., for the purpose of preventing, minimizing, or delaying the development of such diseases or conditions over time. For such prevention purposes, the methods of the present disclosure preferably include continuous, daily administration of the compositions as described herein. Such preventive methods may be directed at pediatrics or others who are at risk of developing glucose intolerance, hyperglycemia, and diabetes.

### Method of Manufacture

The nutritional compositions of the present disclosure may be prepared by any known or otherwise effective manufacturing technique for preparing the selected product form. Many such techniques are known for any given product form such as nutritional liquids, nutritional powders, or nutritional bars and can easily be applied by one of ordinary skill in the nutrition and formulation arts to the nutritional products described herein.

The non-dietary compositions of the present disclosure can likewise be prepared by any known or otherwise effective manufacturing technique for preparing the selected product form. Many such techniques are well known, for example in the pharmaceutical industry, and can be applied by one of ordinary skill in the nutrition and formulation arts to produce forms such as capsules, tablets, caplets, pills, liquids (e.g., suspensions, emulsions, gels, solutions), and so forth, and can easily be applied by one of ordinary skill in those arts to the non-dietary products described herein. As described herein, non-dietary products are those nutritional compositions of the present disclosure that are not dietary products as also defined herein.

Liquid, milk or soy-based nutritional liquids, for example, may be prepared by first forming an oil and fiber blend containing all formulation oils, any emulsifier, fiber and fat-soluble vitamins. Additional slurries (typically a carbohydrate and two protein slurries) are prepared separately by mixing the HMB, carbohydrate and minerals together and the protein in water. The slurries are then mixed together with the oil blend. The resulting mixture is homogenized, heat processed, standardized with any water-soluble vitamins, flavored and the liquid terminally sterilized or aseptically filled or dried, such as by spray drying, to produce a powder.

Other product forms such as nutritional bars may be manufactured, for example, using cold extrusion technology as is known and commonly described in the bar manufacturing art. To prepare such compositions, typically all of the powdered components are dry blended together, which typically includes any proteins, vitamin premixes, certain carbohydrates, and so forth. The fat-soluble components are then blended together and mixed with any powdered premixes. Finally any liquid components are then mixed into the composition, forming a plastic like composition or dough. The resulting plastic mass can then be shaped, without further physical or chemical changes occurring, by cold forming or extrusion, wherein the plastic mass is forced at relatively low pressure through a die, which confers the desired shape. The resultant exudate is then cut off at an appropriate position to give products of the desired weight. If desired, the solid product is then coated, to enhance palatability, and packaged for distribution.

The solid nutritional embodiments of the present disclosure may also be manufactured through a baked application or heated extrusion to produce solid product forms such as cereals, cookies, crackers, and similar other product forms. One knowledgeable in the nutrition manufacturing arts is able to select one of the many known or otherwise available manufacturing processes to produce the desired final product.

The compositions of the present disclosure may also be manufactured by other known or otherwise suitable techniques not specifically described herein without departing from the spirit and scope of the present disclosure. The present embodiments are, therefore, to be considered in all respects as illustrative and not restrictive and that all changes and equivalents also come within the description of the present disclosure. The following non-limiting examples further illustrate the compositions and methods of the present disclosure.

### EXAMPLES

The following Examples provide data and illustrate specific embodiments and/or features of the nutritional compositions and methods of the present disclosure. The Examples are given solely for the purpose of illustration and are not to be construed as limitations, as many variations thereof are possible without departing from the spirit and scope of the disclosure.

### Example 1

In this Example, the effect of (1) HMB and (2) leucine on muscle glucose tolerance was analyzed in an in vivo study.

C57BL/6J mice (Charles River Laboratories, Wilmington Mass.), 21 days post-weaning, were fed either a commercially available pediatric nutritional supplement supplemented with either (1) calcium HMB or (2) leucine, and required to exercise, for nine weeks. Particularly, the test groups (n=6 or 7) were as follows: 1) control group fed Nutrilab® Rodent Pellet Feed (available from Provimi VETCARE® Divn., Netherlands); 2) control group fed pediatric nutritional supplement; 3) test group fed pediatric nutritional supplement supplemented with 5 mg/g leucine; 4) test group fed pediatric nutritional supplement supplemented with 10 mg/g leucine; 5) test group fed pediatric nutritional supplement supplemented with 1.7 mg/g calcium HMB (Abbott Laboratories, Columbus, Ohio); and 6) test group fed pediatric nutritional supplement supplemented with 3.4 mg/g calcium HMB. The exercise consisted of running on a treadmill for 30 minutes, 5 days per week through the duration of the nine weeks.

Following the nine weeks, the mice were kept overnight for fasting and body weight was recorded. A D-glucose (commercially available from Sigma-Aldrich, St. Louis, Mo.) solution was prepared in milli-Q water at a concentration of 200 mg/mL. Before glucose administration, the blood glucose was measured by tail snip using one-touch ultra Glucometer and glucose strips.

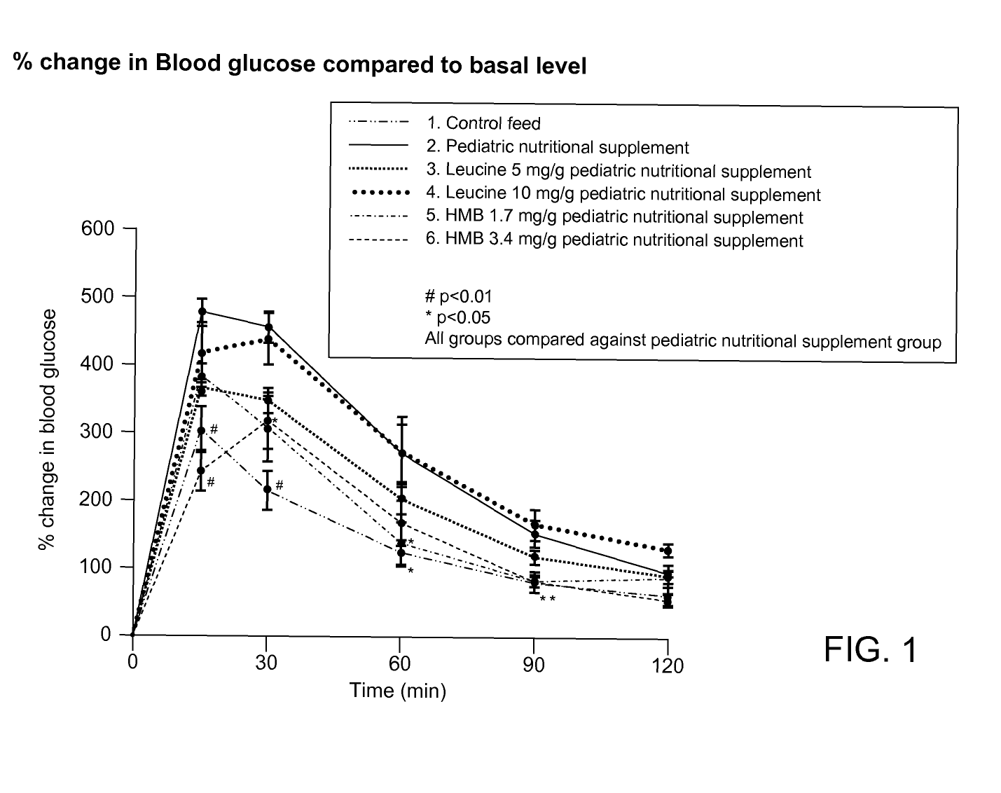

All test groups were orally administered the glucose solution at a concentration of 2 g/Kg body weight. The dose volume was maintained at 10 mL/Kg body weight. After oral glucose administration, blood glucose levels were analyzed at different time points (i.e., 15, 30, 60, 90, and 120 minutes) using Glucometer and glucose strips. The results are shown in FIG. 1.

As shown in FIG. 1, the addition of Calcium HMB at 3.4 mg/g of pediatric nutritional supplement resulted in an improved glucose tolerance as compared to the rodent pellet feed control and the pediatric nutritional supplement control, as well as both leucine groups despite the HMB being present at a lower level in the pediatric nutritional supplement. These results show that administration of Calcium HMB improved glucose tolerance more effectively as compared to leucine.

### Example 2

In this Example, the effect of calcium HMB on lowering glycemic index was evaluated in an in vivo study. Particularly, an acute oral dose of calcium HMB was evaluated to determine whether, if given prior to a glucose load, the dose was able to blunt the initial glucose spike.

Spragley-Dawley (SD) rats (21 days post weaning) (n=7 or 8) were fed an oral dose of calcium HMB at a concentration of: 100 mg/kg (mpk), 300 mpk or 1000 mpk. After thirty minutes, the rats were then orally fed a glucose load consisting of a glucose solution having a concentration of approximately 2 g/kg body weight. A control group (n=4) was not fed the oral dose of calcium HMB prior to the glucose load.

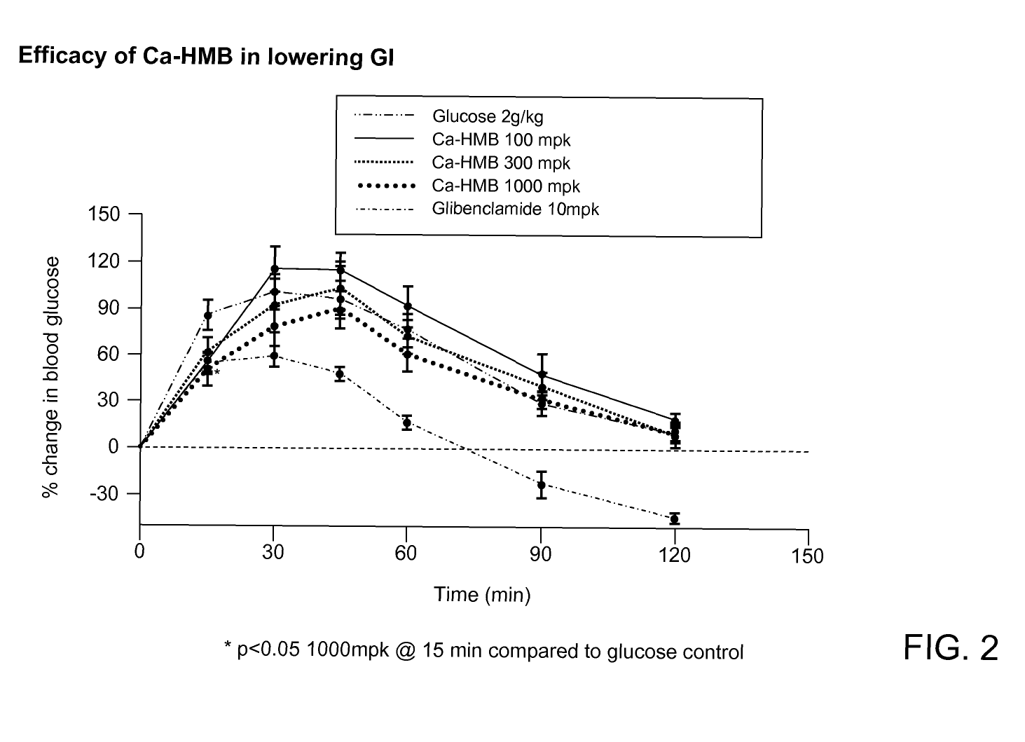

Blood glucose levels of the rats were measured by tail snip using one-touch ultra Glucometer and glucose strips at different time points (0, 15, 30, 45, 60, 90, and 120 minutes). The results are shown in FIG. 2.

As shown in FIG. 2, calcium HMB, at high doses, significantly blunted glucose spike at early time points. Particularly, calcium HMB at a concentration of 1000 mpk significantly reduced glucose spike at 15 minutes post dosing.

### Examples 3-7

Examples 3-7 illustrate pediatric nutritional liquids including calcium HMB in accordance with the present disclosure. The pediatric nutritionals are prepared using a conventional manufacturing process. Amounts in Table 3 below are given in kilograms/1000 kilogram batch unless otherwise noted.

TABLE 3

| | Amount per 1000 KG | | | | | | | | | |
|---|---|---|---|---|---|---|---|---|---|---|
| Ingredients | Ex. 3 | | Ex. 4 | | Ex. 5 | | Ex. 6 | | Ex. 7 | |
| Water | Q.S. | | Q.S. | | Q.S. | | Q.S. | | Q.S. | |
| Calcium HMB | 3.4 | | 3.2 | | 3.0 | | 3.7 | | 4.25 | |
| Corn Maltodextrin | 63.4 | | 63.4 | | 63.4 | | 63.4 | | 63.4 | |
| Sugar | 60.2 | | 60.2 | | 60.2 | | 60.2 | | 60.2 | |
| Milk Protein Concentrate | 24.6 | | 24.6 | | 24.6 | | 24.6 | | 24.6 | |
| High Oleic Safflower Oil | 15.6 | | 15.6 | | 15.6 | | 15.6 | | 15.6 | |
| Soy Oil | 15.3 | | 15.3 | | 15.3 | | 15.3 | | 15.3 | |
| Whey Protein Concentrate | 5.52 | | 5.52 | | 5.52 | | 5.52 | | 5.52 | |
| Medium Chain Triglycerides | 5.46 | | 5.46 | | 5.46 | | 5.46 | | 5.46 | |
| Soy Protein Isolate | 4.92 | | 4.92 | | 4.92 | | 4.92 | | 4.92 | |
| Fructooligosaccharides | 4.45 | | 4.45 | | 4.45 | | 4.45 | | 4.45 | |
| Potassium Hydroxide | 2.41 | | 2.41 | | 2.41 | | 2.41 | | 2.41 | |
| Potassium Citrate | 1.42 | | 1.42 | | 1.42 | | 1.42 | | 1.42 | |
| Magnesium Phosphate | 1.19 | | 1.19 | | 1.19 | | 1.19 | | 1.19 | |
| Flavor | 1.10 | | 1.10 | | 1.10 | | 1.10 | | 1.10 | |
| Microcrystalline Cellulose | 3.00 | | 3.00 | | 3.00 | | 3.00 | | 3.00 | |
| Calcium Phosphate, Tribasic | 860 | g | 860 | g | 860 | g | 860 | g | 860 | g |
| Potassium Chloride | 859 | g | 859 | g | 859 | g | 859 | g | 859 | g |
| Sodium Chloride | 689 | g | 689 | g | 689 | g | 689 | g | 689 | g |
| WSV/TM/UTW Premix | 563 | g | 563 | g | 563 | g | 563 | g | 563 | g |
| Soy Lecithin | 375 | g | 375 | g | 375 | g | 375 | g | 375 | g |
| Monoglycerides | 375 | g | 375 | g | 375 | g | 375 | g | 375 | g |
| Choline Chloride | 337 | g | 337 | g | 337 | g | 337 | g | 337 | g |
| Potassium Phosphate, Monobasic | 313 | g | 313 | g | 313 | g | 313 | g | 313 | g |
| Ascorbic Acid | 227 | g | 227 | g | 227 | g | 227 | g | 227 | g |
| Calcium Carbonate | 214 | g | 214 | g | 214 | g | 214 | g | 214 | g |
| Carrageenan | 150 | g | 150 | g | 150 | g | 150 | g | 150 | g |
| DHA Oil | 118 | g | 118 | g | 118 | g | 118 | g | 118 | g |
| Vitamin Premix | 88.8 | g | 88.8 | g | 88.8 | g | 88.8 | g | 88.8 | g |
| Potassium Phosphate, Dibasic | 70.7 | g | 70.7 | g | 70.7 | g | 70.7 | g | 70.7 | g |
| ARA Oil | 35.1 | g | 35.1 | g | 35.1 | g | 35.1 | g | 35.1 | g |
| Ferrous Sulfate | 34.5 | g | 34.5 | g | 34.5 | g | 34.5 | g | 34.5 | g |
| L-Carnitine | 17.6 | g | 17.6 | g | 17.6 | g | 17.6 | g | 17.6 | g |
| Potassium Iodide | 120 | mg | 120 | mg | 120 | mg | 120 | mg | 120 | mg |

1-18. (canceled)

19. A method of improving glucose tolerance in a pediatric individual in need thereof, the method comprising administering to the pediatric individual a composition comprising an effective amount of beta-hydroxy-beta-methylbutyric acid (HMB), thereby improving glucose tolerance in the pediatric individual.

20. The method according to claim 19, wherein the composition comprises from about 0.1% to about 10% HMB by weight of the composition.

21. The method according to claim 19, wherein the pediatric individual in need thereof is afflicted by or is at risk of developing glucose intolerance, hyperglycemia, or diabetes.

22. The method according to claim 19, wherein the composition is administered daily.

23. The method according to claim 19, wherein the composition is a pediatric nutritional composition comprising calcium beta-hydroxy-beta-methylbutyrate, whey protein, casein protein, soy protein, medium chain triglyceride oil, and fructooligosaccharides.

24. The method according to claim 19, wherein the composition is a pediatric nutritional composition comprising at least one of a protein, a carbohydrate, and a fat, and from about 0.1% to about 10% HMB by weight of the pediatric nutritional composition.

25. The method according to claim 24, wherein the HMB is provided by calcium HMB monohydrate.

26. The method according to claim 25, wherein the pediatric nutritional composition is a liquid composition comprising from about 1% to about 30% protein by weight of the liquid nutritional composition, from about 5% to about 30% carbohydrate by weight of the liquid nutritional composition, and from about 1% to about 30% fat by weight of the liquid nutritional composition.

27. The method according to claim 25, wherein the pediatric nutritional composition is a solid nutritional composition comprising from about 5% to about 30% protein by weight of the solid nutritional composition, from about 20% to about 75% carbohydrate by weight of the solid nutritional composition, and from about 5% to about 35% fat by weight of the solid nutritional composition.

28. A pediatric nutritional composition comprising at least one of a fat, a protein, and a carbohydrate, and from about 0.1% to about 2.0% beta-hydroxy-beta-methylbutyric acid (HMB) by weight of the pediatric nutritional composition.

29. The pediatric nutritional composition of claim 28, wherein the pediatric nutritional composition comprises whey protein, casein protein, soy protein, medium chain triglyceride oil, and fructooligosaccharides, and the HMB is provided by calcium HMB monohydrate.

30. The pediatric nutritional composition of claim 28, wherein the pediatric nutritional composition comprises from about 0.1% to about 0.5% HMB by weight of the pediatric nutritional composition.

31. The pediatric nutritional composition of claim 28, wherein the HMB is provided by calcium HMB monohydrate.

32. The pediatric nutritional composition of claim 28, wherein the pediatric nutritional composition is a liquid nutritional composition and comprises from about 1% to about 10% protein by weight of the liquid nutritional composition; from about 10% to about 20% carbohydrate by weight of the liquid nutritional composition; and from about 1% to about 10% fat by weight of the liquid nutritional composition.

33. The pediatric nutritional composition of claim 32, wherein the liquid nutritional composition comprises from about 5% to about 8% protein by weight of the liquid nutritional composition, from about 15% to about 18% carbohydrate by weight of the liquid nutritional composition, and from about 5% to about 9% fat by weight of the liquid nutritional composition.

34. The pediatric nutritional composition of claim 28, wherein the pediatric nutritional composition is a solid nutritional composition and comprises from about 10% to about 20% protein by weight of the solid nutritional composition, from about 50% to about 70% carbohydrate by weight of the solid nutritional composition, and from about 10% to about 25% fat by weight of the solid nutritional composition.

35. The pediatric nutritional composition of claim 34, wherein the solid nutritional composition comprises from about 12% to about 16% protein by weight of the solid nutritional composition, from about 58% to about 62% carbohydrate by weight of the solid nutritional composition, and from about 15% to about 20% fat by weight of the solid nutritional composition.

36. A method of improving glucose tolerance in an adult individual in need thereof, the method comprising administering to the adult individual a composition comprising an effective amount of beta-hydroxy-beta-methylbutyric acid (HMB), thereby improving glucose tolerance in the adult individual.

37. The method according to claim 36, wherein the adult individual in need thereof is afflicted by or is at risk of developing glucose intolerance, hyperglycemia, or diabetes.

38. The method according to claim 36, wherein the composition comprises from about 0.1% to about 10% HMB by weight of the composition.
